(19) United States
(12) Patent Application Publication
(10) Pub. No.: US 2013/0323167 A1
(43) Pub. Date: Dec. 5, 2013

(54) DETECTING AND TREATING SOLID TUMORS THROUGH SELECTIVE DISRUPTION OF TUMOR VASCULATURE

(71) Applicants: Bert Vogelstein, Baltimore, MD (US); Yuan Qiao, Baltimore, MD (US); Xin Huang, Baltimore, MD (US); Kenneth Kinzler, Baltimore, MD (US); Shibin Zhou, Owings Mills, MD (US); Luis Diaz, Ellicott City, MD (US)

(72) Inventors: Bert Vogelstein, Baltimore, MD (US); Yuan Qiao, Baltimore, MD (US); Xin Huang, Baltimore, MD (US); Kenneth Kinzler, Baltimore, MD (US); Shibin Zhou, Owings Mills, MD (US); Luis Diaz, Ellicott City, MD (US)

(73) Assignee: THE JOHNS HOPKINS UNIVERSITY, Baltimore, MD (US)

(21) Appl. No.: 13/880,608

(22) PCT Filed: Oct. 20, 2011
(86) PCT No.: PCT/US11/57086

§ 371 Date: Jul. 2, 2013

Related U.S. Application Data
(60) Provisional application No. 61/405,241, filed on Oct. 21, 2010.

(51) Int. Cl. A61K 51/10

(52) U.S. Cl. CPC A61K 51/1045
USPC 424/1.49; 424/400; 424/450; 424/1.11

(57) ABSTRACT

Several agents capable of inducing vascular responses akin to those observed in inflammatory processes enhance the accumulation of nanoparticles in tumors. Exemplary vascular-active agents include a bacterium, a pro-inflammatory cytokine, and microtubule-destabilizing drugs. Such agents can increase the tumor to blood ratio of radioactivity by more than 20-fold compared to nanoparticles alone. Moreover, vascular-active agents dramatically improved the therapeutic effect of nanoparticles containing radioactive isotopes or chemotherapeutic agents.

This invention was made with funding from the U.S. government. The U.S. therefore retains certain rights in the invention according to the terms of National Institutes of Health grant CA62924.

### TECHNICAL FIELD OF THE INVENTION

This invention is related to the area of diagnosis and therapy of solid tumors. In particular, it relates to increasing the effectiveness of therapeutic agents and imaging agents.

### BACKGROUND OF THE INVENTION

Wounding results in increased vascular permeability, a process that is markedly enhanced if a wound becomes infected. In response to infection, the mammalian host mobilizes an army of immunoglobulins, complement, white blood cells, and cytokines. To allow this army to engage the enemy, the vascular system at the site of infection must open its gates. This process has been studied in detail and many of the biochemical mechanisms have been identified (1).

Interestingly, it has been said that tumors resemble “unhealed wounds” (2). Accordingly, it is known that the vasculature of tumors is different from that of normal cells, and much effort has gone into exploiting this difference through therapeutic agents like Avastin (3-5). A particularly important phenomenon related to this vascular distinction is referred to as Enhanced Permeability and Retention (EPR) (6). EPR has been identified in many experimental tumor systems and is believed to result from the aberrant tumor vasculature combined with a lack of functional lymphatics in solid tumors. Because of its selectivity for large molecules, EPR has been exploited for therapeutic purposes by using macromolecular drugs or nanoparticles within an appropriate size range (7-10). One notable example is Doxil, a liposomal formulation of doxorubicin, which has been approved for the treatment of human cancers.

There is a continuing need in the art to improve the detection and treatment of solid tumors.

### SUMMARY OF THE INVENTION

According to one aspect of the invention a method is provided to improve delivery of an agent to a solid tumor. A nanoparticle or antibody is administered to an individual who has a solid tumor. The nanoparticle or antibody comprises a therapeutic anti-cancer agent or a detectable imaging agent. A vascular-active permeability entity is also administered to the individual. The vascular-active permeability entity is selected from the group consisting of: a bacterium, a bacterial extract or component, a pro-inflammatory cytokine, and a microtubule destabilizing drug. The amount of the therapeutic anti-cancer or detectable imaging agent delivered to the tumor is thereby increased relative to amount that would be delivered in the absence of the vascular-active entity. Additionally or alternatively, the ratio of the amount of agent delivered to the solid tumor compared to amount delivered to the blood of the individual is increased relative to amount that would be delivered in the absence of the vascular-active entity.

Another aspect of the invention is a kit which comprises a divided or undivided container which contains a vascular-active permeability entity selected from the group consisting of: a bacterium, a bacterial extract or component, a pro-inflammatory cytokine, and a microtubule destabilizing drug; and a nanoparticle or an antibody. The nanoparticle or antibody comprises a therapeutic anti-cancer agent or a detectable imaging agent.

Another aspect of the invention is a composition comprising a vascular-active permeability entity and a nanoparticle or an antibody. The vascular-active permeability entity is selected from the group consisting of: a bacterium, a bacterial extract or component, a pro-inflammatory cytokine, and a microtubule destabilizing drug. The nanoparticle or antibody comprises a therapeutic anti-cancer agent or a detectable imaging agent.

These and other aspects and embodiments which will be apparent to those of skill in the art upon reading the specification provide the art with tools and techniques for improving cancer detection and treatment.

### BRIEF DESCRIPTION OF THE DRAWINGS

FIGS. 1A-1C Inflammatory responses enhance tumor-selective accumulation of radiolabeled antibodies. FIG. 1A, BALB/c mice bearing subcutaneous CT26 tumors were administered C. novyi-NT spores plus ¹²⁵I-labeled liposomase antibody, CD20 antibody, or an IgG control antibody by tail vein injection. The animals were imaged by SPECT/CT 24 hours thereafter. Tumor (Tu), thyroid (Th) and bladder (Bl) are indicated. FIG. 1B and FIG. 1C, Tumor-bearing mice were administered ¹²⁵I-labeled IgG plus C. novyi-NT spores or TNF-α by tail vein injection. For biodistribution analysis (FIG. 1B), mice were sacrificed 48 hours later and percent injected dose per gram of tissue (ID %/g) was determined. Means and s.d. are shown. For imaging study (FIG. 1C), SPECT/CT images were taken at the indicated time points after the injections. Tumor (Tu) is indicated.

FIGS. 2A-2B. Inflammatory responses enhance tumor-selective accumulation of radiolabeled SSLs. BALB/c mice bearing subcutaneous CT26 tumors were administered ¹²⁵I-labeled SSLs plus C. novyi-NT spores or TNF-α by tail vein injection. For biodistribution (FIG. 2A), mice were sacrificed 48 hours later and percent injected dose per gram of tissue (ID %/g) was determined. Means and s.d. are shown. For imaging analysis (FIG. 2B), SPECT/CT images were taken at the indicated time points after the injections. Tumor (Tu), bladder (Bl) and spleen (Sp) are indicated.

FIGS. 3A-3F. TNF-α enhances the antitumor activity of macromolecular drug formulations. Tumor-bearing mice were treated on day 0 with a single dose of the combinations of TNF-α plus ¹³¹I-labeled IgG (FIG. 3A, 3B), Doxil (FIG. 3C, 3D), or ¹³¹I-labeled SSLs (FIG. 3E, 3F), respectively. The therapeutic effects on tumor volume and animal survival are shown. Means and s.e.m. are illustrated. The number of animals used in each experimental arm is shown in parentheses. P values between arms are also shown.

FIGS. 4A-4B. Vascular effect of TNF-α on a brain tumor model. (FIG. 4A) C57BL6 mice bearing orthotopic brain tumors were treated with a single dose of the indicated therapeutic agents 12 days after tumor implantation. The number of animals used in each experimental arm and P values between arms are shown. (FIG. 4B) SPECT-CT images were obtained 48 hours following the indicated treatments, which were performed 25 days following tumor implantation. Transverse, coronal, and sagittal images are shown and tumors indicated by the arrowheads. In this particular animal, two tumor nodules developed along the injection track and both showed tumor accumulation of ¹²⁵I-labeled SSLs when TNF-α was co-administered.

### DETAILED DESCRIPTION OF THE INVENTION

The inventors have found that vascular-active permeability entities are able to increase the amount and/or specificity of delivery to solid tumors. The substance delivered to tumors may be a therapeutic agent or an imaging agent. The substance may include a carrier for the therapeutic or imaging agent or it may be the agent without a carrier. The use of the vascular-active permeability entity increases the amount of agent which is delivered to the solid tumor relative to the amount which was administered to the individual. The use of the vascular-active permeability entity in addition, or alternatively, increases the amount delivered to the solid tumor relative to the amount delivered to the blood of the individual.

Administration of the vascular-active permeability agent and the therapeutic or imaging agent can be accomplished by any means known in the art. Typically, they will be administered by intravenous injections, but other means can be used, including intranasal, intrabronchial, intraductal, intravaginal, oral, intramuscular, subcutaneous, and the like. A single dose may be given of either agent or multiple doses may be administered of one or both agents. Typically the vascular-active permeability agent and the therapeutic or imaging agent are administered at the same time or within 2 hours, 12 hours, 24 hours, 48 hours, 72 hours, 96 hours, 120 hours, 144 hours, or 168 hours or each other. Either agent may be given first.

Nanoparticles as used herein have a size between 10⁻⁵ m and 10⁻⁹ m. The lower limit may be 5×10⁻⁹, 10⁻⁸, 5×10⁻⁸, 10⁻⁷, 5×10⁻⁷, 10⁻⁶, or 5×10⁻⁶ m. The upper limit on size may be 5×10⁻⁶, 10⁻⁶, 5×10⁻⁷, 10⁻⁷, 5×10⁻⁸, 10⁻⁸, or 5×10⁻⁹ m. The nanoparticles may comprise a polymer, carbohydrate, nucleic acid, polypeptide, viral particle, DNA fragment, RNA fragment, a recombinant virus, a recombinant adenovirus, a bacterium, a bacterial spore, liposome, or lipid, for example. The therapeutic or imaging agent may be entrapped, conjugated, encapsulated, or otherwise attached to the nanoparticle.

Antibodies which can be used as a therapeutic or imaging agent or as part of a therapeutic or imaging agent include whole or partial antibodies, such as IgG, ScFv, Fab′, Fab2, and monoclonal antibodies. The antibody may be without limitation bevacizumab (Avastin), cetuximab (Erbitux), trastuzumab (Herceptin), tositumomab, rituximab (Rituxan), ¹³¹I-tositumomab (Bexxar), ¹¹¹In-Zevalin, or ⁹⁰Y-Zevalin, antibodies which are already in clinical use. A therapeutic or imaging agent may be conjugated, fused to, or otherwise attached to the antibody.

The antibodies and nanoparticles may be used as carriers of a therapeutic or imaging agent, including a chemotherapeutic agent, such as doxorubicin, or a prodrug, such as irinotecan (CPT-11). The therapeutic or imaging agent may be a recombinant protein or a peptide. The therapeutic agent may be a toxin, such as botulinum toxin. The therapeutic or diagnostic agent may be an engineered nucleic acid, such as a therapeutic RNA or an aptamer. Any anti-tumor therapeutic agent or imaging agent known in the art may be used, coupled, conjugated, entrapped, or encapsulated by/to an antibody or nanoparticle. An antibody may be a therapeutic agent on its own, without coupling to another moiety.

Examples of types of therapeutic agents and specific examples include, without limitation, alkylating antineoplastic agents, such as cisplatin and carboplatin, oxaliplatin, mechlorethamine, cyclophosphamide, chlorambucil, ifosfamide, antimetabolites such as azathioprine, mercaptopurine, alkaloids, such as vinca alkaloids and taxanes, vincristein, binblastine, vinorelbine, vindesine, podophyllotoxin, doetaxel, topoisomerase inhibitors such as topotecan. amsacrine, etoposide, etoposide phosphate, and teniposide, cytotoxic antibiotics, such as actinomycin, anthracyclines, doxorubicin, daunorubicin, valrubicin, idarubicin, epirubicin, bleomycin, plicamycin, and mitomycin.

A non-limiting list of toxins which may be used as a therapeutic agent include Abrin, Aerolysin, Botulinin toxin A, Botulinin toxin B, Botulinin toxin C1, Botulinin toxin C2, Botulinin toxin D, Botulinin toxin E, Botulinin toxin F, b-bungarotoxin, Caeruleotoxin, Cereolysin, Cholera toxin, Clostridium difficile enterotoxin A, Clostridium difficile cytotoxin B, Clostridium perfringens lecithinase, Clostridium perfringens kappa toxin, Clostridium perfringens perfringolysin O, Clostridium perfringens enterotoxin, Clostridium perfringens beta toxin, Clostridium perfringens delta toxin, Clostridium perfringens epsilon toxin, Conotoxin, Crotoxin, Diphtheria toxin, Listeriolysin, Leucocidin, Modeccin, Nematocyst toxins, Notexin, Pertussis toxin, Pneumolysin, Pseudomonas aeruginosa toxin A, Ricin, Saxitoxin, Shiga toxin, Shigella dysenteriae neurotoxin, Streptolysin O, Staphylococcus enterotoxin B, Staphylococcus enterotoxin F, Streptolysin S, Taipoxin, Tetanus toxin, Tetrodotoxin, Viscumin, Volkensin, and Yersinia pestis murine toxin,

A detectable imaging agent can be coupled, conjugated, entrapped, or encapsulated by/to an antibody or nanoparticle. The imaging agent may be a magnetic material, a photosensitizing agent, a contrast agent, or a radionuclide, for example. The radionuclide may be, for example, Iodine-131 (¹³¹I), Iodine-125 (¹²⁵I), Fluorine-18 (¹⁸F), Gallium-68 (⁶⁸Ga), Copper-64 (⁶⁴Cu), Copper-67 (⁶⁷Cu), Zirconium-89 (⁸⁹Zr), Yttrium-90 (⁹⁰Y), Lutetium-177 (¹⁷⁷Lu), Indium-111 (¹¹¹In), or Technetium-99m (^{99m}Tc). Contrast imaging agents for Magnetic Resonance Imaging (MRI) may include any known in the art including a gadolinium-based contrast agent. Other imaging agents which may be used include Feridex I.V., mangafodipir (Teslascan), a contrast agent for ultrasound, such as a micro-bubble contrast agent, and fluorodeoxyglucose (¹⁸F). After the imaging agent is administered and a suitable time is elapsed for the agent to reach the target tumor, a non-invasive detection technique is performed to generate an image of the tumor in the individual. Suitable techniques include without limitation MRI, ultrasound, PET, and CT scan.

Vascular-active permeability agents are those which increase the amount of a therapeutic or imaging agent which is delivered via the circulation to a tumor. Without limiting the invention to any particular mechanism of action, the agents may act by causing vascular inflammation, or by disrupting the vasculature so that agents of a size which were previously not delivered are delivered, or so that an increase in the amount of an agent of a certain size is delivered. Exemplary vascular-active permeability agents include bacteria (including bacteria which spontaneously infect tumors), such as Clostridium novyi-NT, bacterial spores, a bacterial component, for example lipopolysaccharide (LPS), a vaccine, Coley's toxin, a cytokine, such as tumor necrosis factor-alpha (TNF-α), interferon-gamma (IFN-γ), or interleukin-2 (IL-2), a chemokine, an inducer of cytokine or chemokine expression, e.g., vadimezan (ASA404, DMXAA), and inducer of vascular inflammation, an immune response modifier, a hormone, a pressor agent, such as angiotensin II or adrenaline, a virus, a microtubule interacting agent, such as vinorelbine, combretastatin A4 phosphate (CA4P), HTI-286, or colchicine, a nitric oxide synthase inhibitor, such as L-NAME, L-NNA, or L-NMMA, tumor-localized radiation, and tumor-localized thermotherapy, and high intensity focused ultrasound.

Any combination of named therapeutic or imaging agents with vascular-active permeability entities are specifically contemplated as if each combination were listed separately and explicitly.

Any method known in the art can be used to determine whether the amount of therapeutic agent or imaging agent delivered is increased. These include, without limitation in vivo imaging, biopsy, and agent localization, or tumor response using RESIST criteria. The (1) vascular-active permeability agent and (2) nanoparticles or antibodies with (3) an imaging agent can be used to assess appropriateness of treatment or appropriate dosages of (a) the vascular-active permeability agents and (b) nanoparticles or antibodies with (c) a therapeutic agent. Thus these can be used sequentially or iteratively. Kits may contain the reagents for both assessment and therapeutic uses.

Solid tumors to be treated may be of any type and in any organ of the body of a mammal, such as a farm animal, a pet, a laboratory animal, or a human. These may be in the brain, colon, breasts, prostate, liver, kidneys, lungs, esophagus, head and neck, ovaries, cervix, stomach, colon, rectum, bladder, uterus, testes, and pancreas, as non-limiting examples. The type of tumor may be an adenocarcinoma, a squamous cell carcinoma, or a sarcoma, for example.

The major limitation for most chemotherapeutic agents is their toxicity toward normal tissues, which prohibits the use of doses high enough to eradicate all cancer cells. One approach to address this problem is to develop agents that are delivered to all cells but are preferentially toxic to tumor cells because of the abnormal signaling pathways. This strategy underlies the success of agents such as Gleevec (imatinib) and Iressa (gefitinib) (26, 27). A second approach is to use agents that bind to extracellular molecules present at higher concentrations on the surface of tumor cells, such as Herceptin (trastuzumab) and Erbitux (cetuximab) (28, 29). The third approach takes advantage of the abnormal vasculature present in tumors, allowing preferential accumulation of nanoparticles (the EPR effect) (6, 30). Though all approaches have merit, the third has the advantage that virtually any drug, including a wealth of clinically approved agents, can in theory be made more effective by its incorporation into nanoparticles of appropriate sizes. The ability to use agents that are already clinically approved poses many practical advantages with respect to the performance of clinical trials and the duration of the drug approval process.

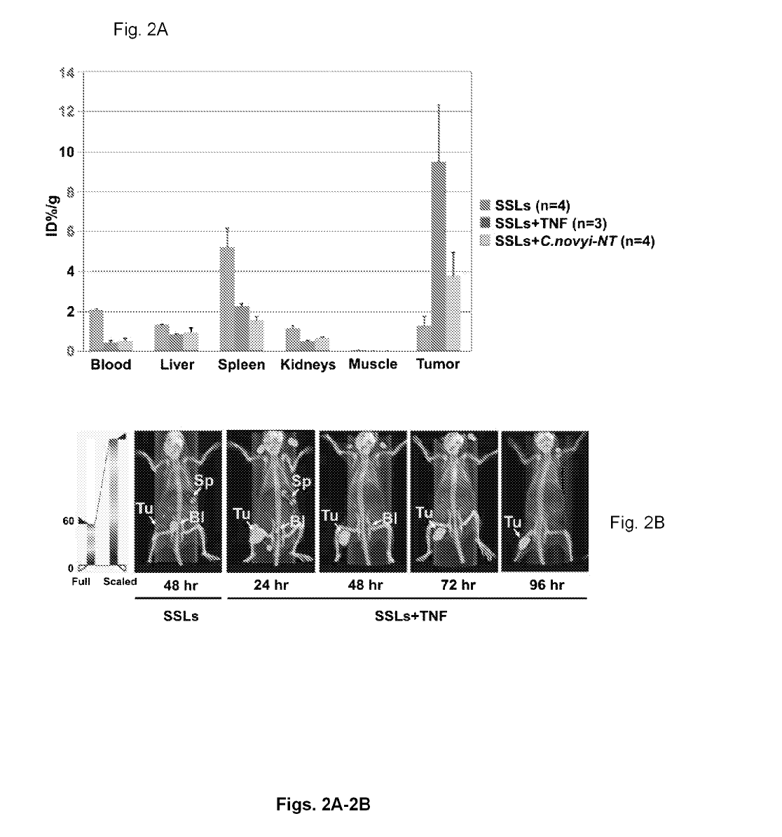

In this work, we have attempted to enhance the third approach through pharmacologic manipulation of the abnormal vasculature already present in tumors. We show that Enhanced EPR (E²PR). can dramatically increase the tumor: blood ratio of nanoparticles, as assessed by both imaging and therapeutic response. It is worth noting that even a small difference in the intratumoral concentration of an agent can make a large difference in therapeutic effect (31). In the studies described here, E²PR led to a tumor: blood ratio of more than 22-fold (FIG. 2A).

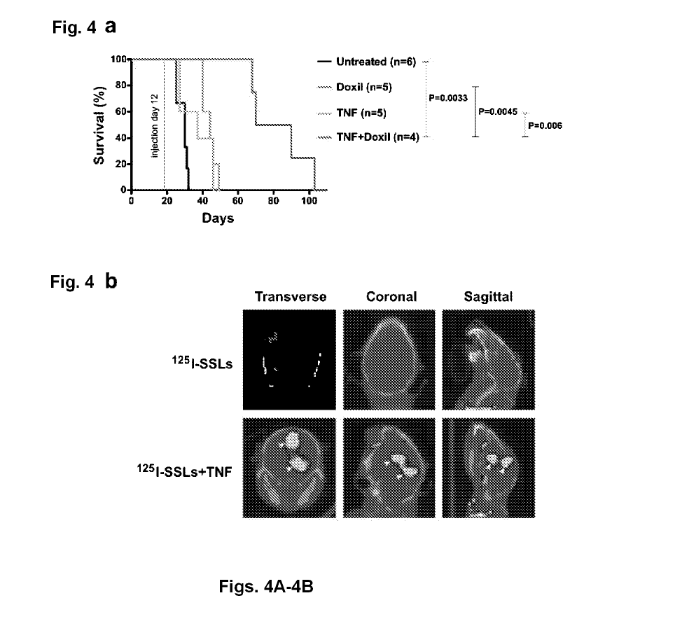

We were particularly encouraged with the results in the GBM model. This tumor type in humans is highly recalcitrant to conventional therapies, leading to a dismal prognosis for patients with this disease. The blood-brain barrier is at least partly to blame for the limited efficacy of chemotherapy (32). We found that TNF-α treatment could help breach the blood-brain barrier and result in major accumulations of ¹²⁵I-labeled sterically stabilized liposomal nanoparticles (SSLs) in the orthotopically implanted brain tumors as well as significantly prolong the survival of the tumor-bearing animals (FIG. 4). As the mouse cranial cavity is small, murine brain tumors are particularly difficult to treat as even a minimal amount of growth of a pre-existing tumor is lethal.

Our results suggest a way to improve the therapeutic efficacy of conventional and novel drugs by incorporating them into nanoparticles and injecting them together with vascular-active agents such as TNF-α. The approach is versatile, as it should be practicable with a variety of nanoparticle formulations as well as with diverse chemical and radioactive agents.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### Example 1

### Materials and Methods

### Cell Lines

CT26 (CRL-2638) murine colorectal adenocarcinoma cells were purchased from the American Type Culture Collection (ATCC) and grown in McCoy's 5A Medium (Invitrogen) supplemented with 10% Fetal Bovine Serum (FBS, HyClone) at 37° C. with 5% CO2. GL261 glioma cells were kindly provided by Dr. Michael Lim (Johns Hopkins University, Baltimore) and maintained in DMEM media (ATCC) supplemented with 10% FBS.

### Reagents

Bolton-Hunter reagent (BH, N-succinimidyl-3-(4-hydroxyphenyl)-propionate) and TNF-α (mouse, recombinant) were purchased from Sigma-Aldrich. Radioiodines (Sodium 125- or 131-iodide) were purchased from MP Biomedicals and Nordion, respectively. IODO-GEN was purchased from Pierce. Mouse monoclonal IgG1 isotype control antibody (ab18447) and CD20 antibody (ab8237) were purchased from Abcam. PEGylated liposomal doxorubicin) (DOXIL® was purchased from Tibotec Therapeutics. Hydrogenated Chicken Egg L-α-Phosphatidylcholine (HEPC), 1,2-Distearoyl-sn-Glycero-3-Phosphoethanolamine-N-[Methoxy(Polyethylene glycol)-2000] (DSPE-PEG2000) and Cholesterol (Chol) were purchased from Avanti Polar Lipids. C. novyi-NT spores were prepared as previously described (11).

### Animal Models

All animal experiments were overseen and approved by the Animal Welfare Committee of Johns Hopkins University, and were in compliance with the University standards. For the subcutaneous tumor model, female, six to eight week-old BALB/c mice (Harlan Breeders) were used. Five million CT26 cells were injected subcutaneously into the right flank of each mouse and allowed to grow for ˜10 days before randomization, group assignment, and treatment. C. novyi-NT spores were administered by a bolus tail vein injection of 300 million spores suspended in 0.2 mL of phosphate buffered saline, pH 7.5 (PBS). Cytotoxic anticancer agents were administered 16 hours later via the same route. TNF-α was reconstituted freshly before administration in doubly-distilled H2O at 100 ng/mL and diluted into 0.1% (w/v) BSA in PBS at a final concentration of 10 ng/mL. Cytotoxic agents were injected within a few minutes thereafter. Tumor volume was calculated as length×width×0.5. For the orthotopic brain tumor model, female C57BL6 mice, 5-6 weeks of age, were purchased from the NCI-Frederick. Mice were anesthetized via intraperitoneal injection of 60 μL of a stock solution containing ketamine hydrochloride (75 mg/kg, Abbot Laboratories), xylazine (Xyla-ject®, 7.5 mg/kg, Phoenix Pharmaceutical), and ethanol (14.25%) in a sterile 0.9% NaCl solution. Following a 1-cm midline scalp incision, a 1-mm burr hole was placed over the right frontal bone, with its center 2 mm lateral to the sagittal suture and 1 mm anterior to the coronal suture. On a stereotactic frame, a sterile needle loaded with 20,000 GL261 cells was placed at a depth of 3 mm below the dura and the cells were injected slowly at a rate of 1 μL/minute. Afterwards, the animal was removed from the frame and the scalp incision closed with surgical staples. On day 12 post implantation of the tumor cells, a significant tumor was formed and 1 μg of mouse recombinant TNF-α or 100 μL of Doxil at 20 mg/kg, or both, were administered intravenously through the tail vein. Animals were monitored for potential side effects following drug administration. Animals were observed daily for any signs of deterioration, neurotoxicity, or movement disorders. They were inspected for signs of pain and distress, as per the Johns Hopkins Animal Care and Use Guidelines. If the symptoms persisted and resulted in debilitation, the moribund animals were euthanized. The brain and other organs were dissected and placed in formalin for further pathological studies.

### Liposomase Antibody

Three peptides (JHU009A: CNVDLQQKLIEN; JHU009B: CYPEWGTKDENGNIRK; JHU009C: CDMAQMLRNLPVTE) were used to immunize the mice for generating antibodies against C. novyi-NT liposomase (A&G Pharmaceutical). After screening ˜500 hybridoma clones by ELISA, one clone (JHU009-5F5) specific to the JHU009C peptide was eventually selected for the imaging study. The affinity and specificity of the JHU009-5F5 mAb were also confirmed by both ELISA and western blot analyses against purified liposomase protein (12).

### Radioiodination of Antibodies

Typically, 20 μg of purified antibody in 100 μL of PBS was added to an iodogen-coated vial. Sodium 125- or 131-iodide was then added to the vial at 2 to 5 mCi in 2 to 5 μL of 0.1 M NaOH, pH 10. The reaction was then incubated for 10 minutes at room temperature before purification on a PBS-equilibrated Sephadex G-25 desalting column (Amersham Biosciences) to remove unincorporated radioiodine. The radiochemical yield was typically 30% to 40%. The radiochemical purity was at least 95% as determined by thin-layer chromatography. Antibodies were labeled within 24 hours of use and stored in PBS at 4° C. after labeling and purification.

### Preparation of Liposomes

A mixture of HEPC:Chol:DSPE-PEG2000 at a molar ratio of 50:45:5 was solubilized in chloroform, followed by drying to a thin film under rotary evaporation and then under vacuum for 2 hours. The film was hydrated with arginine solution (80 mmol/L) in 4-(2-hydroxyethyl)-piperazine-1-sulphonic acid (HEPES, 80 mmol/L, pH 8.0) and submerged in a 65° C. sonication bath (Bransonic) to form Large Multilamellar Vesicles (MLVs). This lipid suspension was extruded 10 times through a double stack of 0.1 μm Nuclepore filters (Whatman) using a Lipex Thermobarrel Extruder (Northern Lipids). The resulting colloidal suspension of Single Unilamellar Vesicles (SUV) was dialyzed against 150 mmol/L phosphate buffer (pH 5.6) at 4° C. to exchange the external milieu of the liposomes and then filter-sterilized. The mean size of the SUVs was ˜100 nm in diameter and polydispersity index ˜0.1 as determined by quasi-elastic light scattering using a Malvern Zetasizer 3000 (Malvern).

### Radioiodination of Bolton-Hunter Reagent

Bolton-Hunter reagent (BH, N-hydroxysuccinimide (NHS) ester of HPPA) was labeled with sodium 125- or 131-iodide by the chloramine-T method and purified by solvent extraction. Briefly, 50 μL of chloramine T (4 mg/mL in phosphate buffer) and 3.7 to 37 MBq (0.1-1.0 mCi) of ¹²⁵I—NaI or ¹³¹I—NaI were added to 2 μL of BH freshly solubilized in anhydrous dioxin (0.5 mg/mL). Iodination was achieved by incubation at room temperature for approximately 15 sec and then 400 μL of 100 mmol/L phosphate buffer (pH 7.4) was added. The radiolabeled BH was immediately extracted with 500 μL of toluene and the organic phase was removed and transferred to a glass tube. For the encapsulation of the reagent into liposomes, the organic solvent was evaporated using a dry nitrogen stream before adding the liposome suspension.

Encapsulation of the Iodinated Reagents into the Liposomes

For the chemical entrapment of the iodinated BH, arginine-containing liposomes were incubated for 30 min at 37° C. with ¹²⁵I—BH. The labeling efficiency was determined by counting the liposome suspension before and after chromatography on a PD-10 column (GE Healthcare) (13). The radiochemical yield was typically 50% to 70%.

### Biodistribution Assay

CT26-bearing BALB/c mice were injected via the tail vein with 50 μCi of ¹²⁵I-liposomes or ¹²⁵I-IgG1. Three to four mice in each experimental arm were sacrificed by cervical dislocation at 48 hours post injection. The liver, spleen, kidneys, muscle, and tumor were quickly removed as was ˜0.1 mL of blood. The organs and blood were weighed and their radioactivity was measured with an automated gamma counter (1282 Compugamma CS, Pharmacia/LKB Nuclear). The percent injected dose per gram of tissue (ID %/g) was calculated by comparison with samples of a standard dilution of the initial dose. All measurements were corrected for decay.

### SPECT-CT Imaging

BALB/c mice bearing subcutaneous CT26 tumor or C57BL6 mice bearing orthotopic GL261 brain tumor were injected intravenously with 37.5 MBq (1 mCi) of either ¹²⁵I-IgG1 or ¹²⁵I-SSLs in saline. The mice were positioned on the X-SPECT (Gamma Medica-Ideas) gantry and scanned using two low-energy, high resolution pinhole collimators (Gamma Medica-Ideas) rotating through 360° in 6° increments for 40 seconds per increment Immediately following SPECT acquisition, the mice were scanned by CT (X-SPECT) over a 4.6 cm field of view using a 600 mA, 50 kV beam. Data were reconstructed using the ordered subsets-expectation maximization algorithm. The SPECT and CT data were then coregistered using the instrument supplied software and displayed using AMIDE (http://amide.sourceforge.net/) or Amira software (Visage Imaging).

### Statistical Analysis

The statistical significance of percent survival between different experimental arms was determined by Long-rank analysis.

### Example 2

### Bacterial Infection Enhances Antibody Accumulation in Experimental Tumors

The research described in this work was stimulated by unexpected observations made through the investigation of C. novyi-NT, an attenuated anaerobic bacterial strain that can infect experimental tumors (11). This infection often leads to eradication of the internal hypoxic regions of tumors but leaves the oxygenated rim of the tumors intact. C. novyi-NT secretes an enzyme called liposomase at high levels in the infected tumors (12, 14). We hypothesized that a radiolabeled anti-liposomase antibody would synergize with C. novyi-NT by binding to liposomase secreted by the bacteria, thereby eradicating the oxygenated tumor rim through β-particle irradiation. A monoclonal antibody against liposomase was generated and used to evaluate this hypothesis (see Methods).

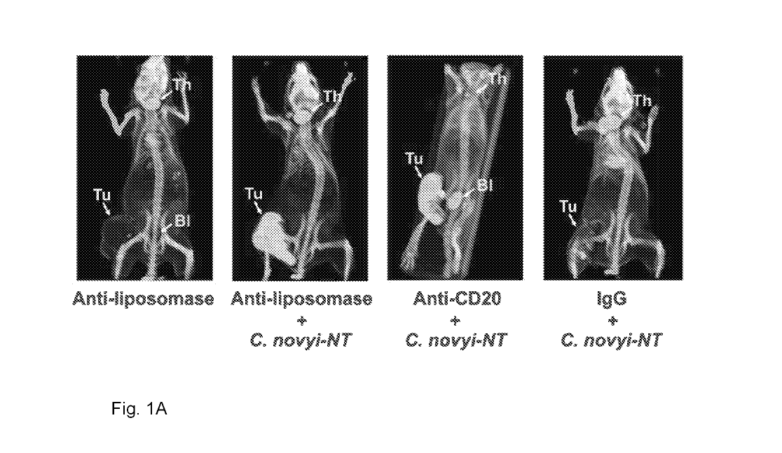

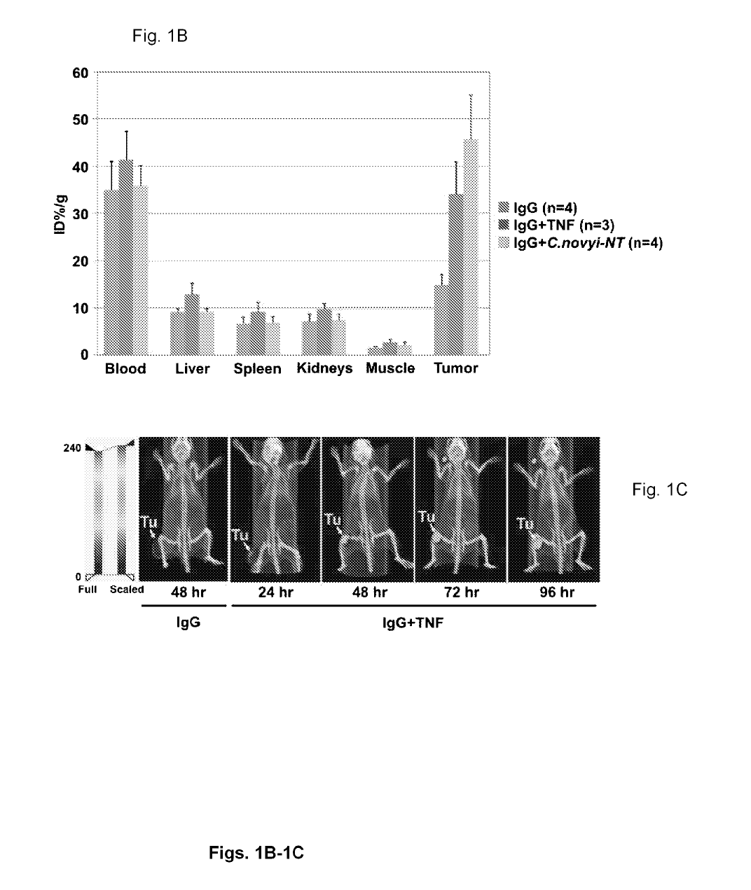

Mice bearing subcutaneous CT26 tumors were intravenously injected with C. novyi-NT spores together with the radiolabeled anti-liposomase antibody or with a similarly labeled IgG control antibody. The anti-liposomase antibody was highly enriched in the tumors infected with C. novyi-NT but not in uninfected tumors (FIG. 1A). Surprisingly, however, the radiolabeled IgG control antibody was also enriched in the C. novyi-NT infected tumors, albeit to a lesser extent (FIG. 1A). Biodistribution analyses showed that the level of radioactivity in the tumor was four-fold higher than that in most normal tissues (FIG. 1B).

To further confirm that the accumulation in the tumors was not antibody-specific, we repeated the experiment with another antibody generated against human CD20, a B-cell antigen. The partially humanized version of this antibody, Rituximab, has been marketed for the treatment of B cell lymphoma and chronic lymphocytic leukemia (15, 16). Systemically administered anti-CD20 antibody was also enriched in the tumor if the animal was simultaneously injected with C. novyi-NT spores (FIG. 1A).

### Example 3

### Bacterial Infection and Pro-Inflammatory Cytokine Both Enhance Tumor-Selective Accumulation of Macromolecular Drug Formulations

We reasoned that the inflammatory response to the bacterial infection led to an increased vascular permeability, resulting in the preferential antibody accumulation at the infected tumor site. We therefore sought to identify a pro-cytokine that might mimic the effects of C. novyi-NT. Among those considered, Tumor Necrosis Factor-α (TNF-α) was of particular interest as this cytokine has been identified as the serum factor responsible for endotoxin-induced vascular permeabilization (17, 18). Furthermore, a similar hemorrhagic necrosis in tumors is observed following systemic administration of either TNF-α or C. novyi-NT spores (11, 17). Based on these parallels, we repeated the protocol described above, substituting systemically-administered TNF-α for C. novyi-NT spores. When CT26 tumor-bearing mice were injected with murine TNF-α and radiolabeled murine IgG, significant IgG accumulation was observed in the tumors but not in the normal tissues (FIGS. 1B and C). A time course study revealed that the IgG tumor accumulation progressed slowly and peaked between 72 and 96 hours after injection (FIG. 1C).

The effect of vascular-active agents on tumor vasculature will henceforth be referred to as Enhanced EPR (E²PR). Sterically stabilized liposomal nanoparticles (SSLs) of ˜100 nm in diameter have been shown to be susceptible to the EPR effect (8). To evaluate whether such liposomes were susceptible to E²PR, we fabricated radioactive liposomes using a Bolton-Hunter (BH) reagent-based iodination strategy (13). Iodinated BH reagent labels proteins by forming amide bonds with free amino groups such as those present on arginine (19). SSLs were loaded with arginine at low pH and then the loaded SSLs were incubated with ¹²⁵I-labeled BH reagent. The ¹²⁵I—BH reagent passed through the lipid bilayer but was unable to exit after covalent binding to the arginine because of the latter's positive charge. We were thus able to achieve a very high concentration of radioactivity within the SSLs while avoiding prolonged exposure to the radioactivity during the preparation.

¹²⁵I-labeled SSLs were intravenously injected into tumor-bearing mice in combination with either C. novyi-NT or TNF-α. Both C. novyi-NT and TNF-α treatments significantly augmented the selective retention of ¹²⁵I within tumors (FIG. 2). Furthermore, the radioactivity in normal tissues was markedly lower compared to the animals treated with ¹²⁵I-labeled SSLs without TNF-α or C. novyi-NT (FIG. 2A).

Thus, the tumor-to-blood radio of radioactivity following TNF-α treatment was as high as 22-fold, far higher than achieved with radiolabeled IgG (compare FIG. 2A to FIG. 1B). SPECT/CT also revealed that the kinetics of tumor accumulation was different with radiolabeled SSLs than with IgG: SSL accumulation peaked at 24 hours, 48-72 hours earlier than IgG.

Like EPR, the effect of E²PR is particle size-dependent. In contrast to ¹²⁵I-labeled SSLs, tumor retention of ¹²⁵I-labeled arginine (the substrate of ¹²⁵I labeling in SSLs) is not affected by TNF-α. However, at the other end of the size spectrum, ¹²⁵I-labeled C. novyi-NT spores (˜1 μm in diameter (20)) are highly enriched in tumors only when combined with TNF-α (data not shown). Thus, E²PR appears to reflect a more substantial vascular disruption than EPR: while EPR favors accumulation of nanoparticles in the range around 100 nm (8), E²PR extends that range to >1 μm.

To determine whether the accumulation was dependent on the volume of the tumor, we injected TNF-α plus ¹²⁵I-labeled IgG or ¹²⁵I-labeled SSLs into animals with a small subcutaneous tumor on one flank and a large tumor on the other flank. SPECT/CT showed retention of radioactivity in both tumors. We also tested the relative timing of injection of TNF-α and ¹²⁵I-labeled SSLs. Though TNF-α and SSLs were administered jointly in the experiments recorded above, we found that similar results were obtained when TNF-α was administered within 12 hours after SSLs. Conversely, E²PR was not observed when TNF-α was administered 6 hours prior to SSL administration (data not shown).

Microtubule-interacting agents are also able to disrupt the tumor vasculature (21). We therefore determined whether such agents could induce E²PR. Combretastatin A4P(CA4P) and vinorelbine are microtubule-interacting agents with completely different structures and modes of interaction with microtubules (22, 23). Injection of either resulted in E²PR, though not as impressively as TNF-α.

### Example 4

### TNF-α and Macromolecular Drug Formulations Synergize in the Treatment of Experimental Tumors

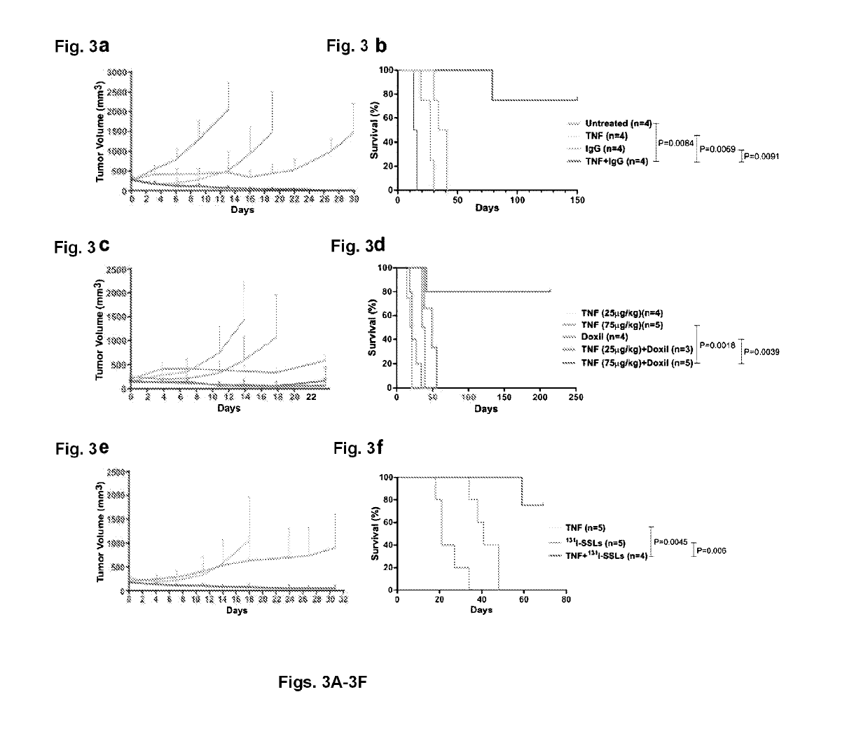

We next investigated whether the E²PR could be translated into therapeutic gain. Mice bearing fully developed CT26 tumors were treated by simultaneous i.v. injections of TNF-α plus Doxil (10 mg/kg) or radiolabeled IgG. ¹³¹I rather than ¹²⁵I was chosen for radiolabeling in light of the type of ionizing radiation required for a radiotherapeutic effect. Although treatment with Doxil or ¹³¹I-labeled IgG in the absence of TNF-α retarded tumor growth and prolonged animal survival, the tumors always grew back (FIGS. 3A and B). When combined with TNF-α, however, a single administration of these agents led to complete tumor regression in all animals and long-term cures in more than 75% of them. When a lower dose (25 ng/kg) of TNF-α was used, none of the treated animals were cured, although prolonged survival was observed. It is important to note that humans tolerate multiple injections (3 infusions/week) of a dose comparable to the highest dose of TNF-α we used (24). We also tested SSLs containing ¹³¹I, generated using the chemical trapping approach described above. While ¹³¹I-labeled SSLs alone retarded tumor growth, complete tumor regression and cures were only observed when they were used in combination with TNF-α (FIG. 3C).

Finally, we evaluated the therapeutic potential of E²PR in a murine model of glioblastoma multiforme (GBM). When implanted orthotopically, the brain tumor cell line GL261 forms very aggressive tumors, killing animals within about a month (FIG. 4A). At the histologic level, these tumors are very similar to human GBM, manifesting an infiltrative growth pattern, necrosis and neovascularization (25). Following stereotactic injection of GL261 cells into the frontal lobe, brain tumors were allowed to grow to substantial size, then ¹²⁵I-labeled SSLs with or without TNF-α were administered. Tumor accumulation of the radiolabeled SSLs was only observed in TNF-α treated animals (FIG. 4B). Mice with similar tumors were injected with Doxil, either with or without TNF-α. The combination clearly had a therapeutic benefit, prolonging survival up to 103 days even in this highly challenging pre-clinical model (FIG. 4A). Both Doxil and TNF-α showed limited therapeutic benefit when used as single agents, with no animal surviving beyond 50 days following tumor implantation.

### REFERENCES

The disclosure of each reference cited is expressly incorporated herein.
1. Cotran R S. Inflammation: historical perspectives. In: Gallin J I, Snyderman, R., editor. Inflammation: basic principles and clinical correlates. 3th ed. Philadelphia: Lippincott Williams & Wilkins 1999. p. 5-10.
2. Dvorak H F. Tumors: wounds that do not heal. Similarities between tumor stroma generation and wound healing. N Engl J Med 1986; 315: 1650-9.
3. Jain R K, Duda D G. Vascular and Interstitial Biology of Tumors. In: Abeloff M D, Armitage J O, Niederhuber J E, Kastan M B, McKenna W G, editors. ABELOFF'S CLINICAL ONCOLOGY. 4th ed. Philadelphia: Churchill Livingstone Elsevier; 2008. p. 105-24.
4. Ferrara N, Hillan K J, Gerber H P, Novotny W. Discovery and development of bevacizumab, an anti-VEGF antibody for treating cancer. Nat Rev Drug Discov 2004; 3: 391-400.
5. Hurwitz H, Fehrenbacher L, Novotny W, et al. Bevacizumab plus irinotecan, fluorouracil, and leucovorin for metastatic colorectal cancer. N Engl J Med 2004; 350: 2335-42.
6. Matsumura Y, Maeda H. A new concept for macromolecular therapeutics in cancer chemotherapy: mechanism of tumoritropic accumulation of proteins and the antitumor agent smancs. Cancer Res 1986; 46: 6387-92.
7. Maeda H, Fang J, Inutsuka T, Kitamoto Y. Vascular permeability enhancement in solid tumor: various factors, mechanisms involved and its implications. Int Immunopharmacol 2003; 3: 319-28.
8. Drummond D C, Meyer O, Hong K, Kirpotin D B, Papahadjopoulos D. Optimizing liposomes for delivery of chemotherapeutic agents to solid tumors. Pharmacol Rev 1999; 51: 691-743.
9. Torchilin V P. Micellar nanocarriers: pharmaceutical perspectives. Pharm Res 2007; 24: 1-16.
10. Maeda H, Bharate G Y, Daruwalla J. Polymeric drugs for efficient tumor-targeted drug delivery based on EPR-effect. Eur J Pharm Biopharm 2009; 71: 409-19.
11. Dang L H, Bettegowda C, Huso D L, Kinzler K W, Vogelstein B. Combination bacteriolytic therapy for the treatment of experimental tumors. Proc Natl Acad Sci U S A 2001; 27: 27.
12. Cheong I, Huang X, Bettegowda C, et al. A bacterial protein enhances the release and efficacy of liposomal cancer drugs. Science 2006; 314: 1308-11.
13. Mougin-Degraef M, Jestin E, Bruel D, et al. High-activity radio-iodine labeling of conventional and stealth liposomes. J Liposome Res 2006; 16: 91-102.
14. Bettegowda C, Huang X, Lin J, et al. The genome and transcriptomes of the anti-tumor agent Clostridium novyi-NT. Nat Biotechnol 2006; 24: 1573-80.
15. Coiffier B. Rituximab therapy in malignant lymphoma. Oncogene 2007; 26: 3603-13.
16. Jaglowski S M, Byrd J C. Rituximab in chronic lymphocytic leukemia. Semin Hematol; 47: 156-69.
17. Carswell E A, Old L J, Kassel R L, Green S, Fiore N, Williamson B. An endotoxin-induced serum factor that causes necrosis of tumors. Proc Natl Acad Sci U S A 1975; 72: 3666-70.
18. Brett J, Gerlach H, Nawroth P, Steinberg S, Godman G, Stern D. Tumor necrosis factor/cachectin increases permeability of endothelial cell monolayers by a mechanism involving regulatory G proteins. J Exp Med 1989; 169: 1977-91.
19. Bolton A E, Hunter W M. The labelling of proteins to high specific radioactivities by conjugation to a ¹²⁵I-containing acylating agent. Biochem J 1973; 133: 529-39.
20. Plomp M, McCaffery J M, Cheong I, et al. Spore coat architecture of Clostridium novyiNT spores. J Bacteriol 2007; 189: 6457-68.
21. Schwartz E L. Antivascular actions of microtubule-binding drugs. Clin Cancer Res 2009; 15: 2594-601.
22. Tozer G M, Kanthou C, Parkins C S, Hill S A. The biology of the combretastatins as tumour vascular targeting agents. Int J Exp Pathol 2002; 83: 21-38.
23. Holwell S E, Hill B T, Bibby M C. Anti-vascular effects of vinflunine in the MAC 15A transplantable adenocarcinoma model. Br J Cancer 2001; 84: 290-5.
24. Schiller J H, Storer B E, Witt P L, et al. Biological and clinical effects of intravenous tumor necrosis factor-alpha administered three times weekly. Cancer Res 1991; 51: 1651-8.
25. Ausman J I, Shapiro W R, Rall D P. Studies on the chemotherapy of experimental brain tumors: development of an experimental model. Cancer Res 1970; 30: 2394-400.
26. Druker B J. Imatinib as a paradigm of targeted therapies. Adv Cancer Res 2004; 91: 1-30.
27. Mok T S, Wu Y L, Thongprasert S, et al. Gefitinib or carboplatin-paclitaxel in pulmonary adenocarcinoma. N Engl J Med 2009; 361: 947-57.
28. Slamon D J, Leyland-Jones B, Shak S, et al. Use of chemotherapy plus a monoclonal antibody against HER2 for metastatic breast cancer that overexpresses HER2. N Engl J Med 2001; 344: 783-92.
29. Cunningham D, Humblet Y, Siena S, et al. Cetuximab monotherapy and cetuximab plus irinotecan in irinotecan-refractory metastatic colorectal cancer. N Engl J Med 2004; 351: 337-45.
30. Seynhaeve A L, Hoving S, Schipper D, et al. Tumor necrosis factor alpha mediates homogeneous distribution of liposomes in murine melanoma that contributes to a better tumor response. Cancer Res 2007; 67: 9455-62.
31. Frei E, 3rd, Canellos G P. Dose: a critical factor in cancer chemotherapy. Am J Med 1980; 69: 585-94.
32. Muldoon L L, Soussain C, Jahnke K, et al. Chemotherapy delivery issues in central nervous system malignancy: a reality check. J Clin Oncol 2007; 25: 2295-305.

1. A method to improve delivery of an agent to a solid tumor, comprising:
administering a nanoparticle or an antibody to an individual who has a solid tumor, wherein the nanoparticle and the antibody comprise a therapeutic anti-cancer agent or a detectable imaging agent;
administering to the individual a vascular-active permeability entity selected from the group consisting of: a bacterium, a bacterial extract or component, and a microtubule destabilizing drug, whereby amount of the agent delivered to the tumor is increased relative to amount that would be delivered in the absence of the vascular-active entity or whereby ratio of agent delivered to tumor versus blood is increased relative to amount that would be delivered in the absence of the vascular-active entity.

2. (canceled)

3. The method of claim 1 wherein the nanoparticle is administered and it is a sterically stabilized liposome.

4. The method of claim 1 wherein the nanoparticle is administered and it is between 6 nm and 1 um in diameter (6×10⁻⁹ and 1×10⁻⁶ m).

5. The method of claim 1 wherein a composition of nanoparticles is administered and the average size of the nanoparticles in the composition is between 6 nm and 1 um in diameter (6×10⁻⁹ and 1×10⁻⁶ m).

6. (canceled)

7. (canceled)

8. (canceled)

9. The method of claim 1 wherein the antibody or nanoparticle comprise a detectable imaging agent and the method further comprises performing a non-invasive detection technique to generate an image of the tumor in the individual.

10. The method of claim 1 wherein the vascular-active permeability entity is a bacterium, bacterial extract or component, or bacterial spore.

11. (canceled)

12. The method of claim 1 wherein the vascular-active permeability entity is selected from the group consisting of: lipopolysaccharide, a pro-inflammatory cytokine, TNF-alpha, vinorelbine, and Combrestatin A4P.

13. (canceled)

14. (canceled)

15. (canceled)

16. (canceled)

17. (canceled)

18. (canceled)

19. The method of claim 1 wherein the nanoparticle or antibody comprises a radioisotope.

20. (canceled)

21. (canceled)

22. (canceled)

23. The method of claim 1 wherein the vascular-active permeability entity is administered within 12 hours of administration of the nanoparticle.

24. The method of claim 1 wherein the vascular-active permeability entity is administered within 0 to 7 days after administration of the nanoparticle.

25. The method of claim 1 wherein the solid tumor is selected from the group consisting of: a brain tumor, a carcinoma, a sarcoma, an adenocarcinoma, and a squamous cell carcinoma.

26. (canceled)

27. (canceled)

28. (canceled)

29. (canceled)

30. A kit comprising in a divided or undivided container:
a vascular-active permeability entity selected from the group consisting of: a bacterium, a bacterial extract or component, and a microtubule destabilizing drug; and
a nanoparticle or an antibody which comprises a therapeutic anti-cancer agent or a detectable imaging agent.

31. (canceled)

32. (canceled)

33. (canceled)

34. (canceled)

35. (canceled)

36. The kit of claim 29 wherein the kit comprises a nanoparticle and the nanoparticle is between 6 nm and 1 μm in diameter (6×10⁻⁹ and 1×10⁻⁶ m).

37. The kit of claim 29 which comprises a composition of nanoparticles and the average size of the nanoparticles in the composition is between 6 nm and 1 μm in diameter (6×10⁻⁹ and 1×10⁻⁶ m).

38. A composition comprising:
a vascular-active permeability entity selected from the group consisting of: a bacterium, a bacterial extract or component, and a microtubule destabilizing drug; and
a nanoparticle or an antibody which comprises a therapeutic anti-cancer agent or a detectable imaging agent.

39. (canceled)

40. The composition of claim 37 wherein the composition comprises a nanoparticle, and the nanoparticle is between 6 nm and 1 μm in diameter (6×10⁻⁹ and 1×10⁻⁶ m).

41. The composition of claim 37 which comprises a plurality of nanoparticles and the average size of the nanoparticles in the composition is between 6 nm and 1 μm in diameter (6×10⁻⁹ and 1×10⁻⁶ m).
